(19) United States
(12) Patent Application Publication
(10) Pub. No.: US 2004/0072902 A1
(43) Pub. Date: Apr. 15, 2004

(54) Methods for inducing apoptosis and inhibiting proliferation in cancer cells

(76) Inventors: Thomas E. Adrian, Chicago, IL (US); Peter D. Collin, Sunset, ME (US)

Correspondence Address: Peter Rogalskj, Rogalskj & Weyand, PO Box 44, Livonia, NY, 14487-0044, US

(21) Appl. No.: 10/451,555

(22) PCT Filed: Dec. 24, 2001
(86) PCT No.: PCT/US01/49689

(51) Int. Cl. A61K 31/202

(52) U.S. Cl. 514/560

(57) ABSTRACT

Disclosed are methods of decreasing proliferation of adenocarcinoma cells, or of inducing apoptosis of adenocarcinoma cells, or of inducing differentiation of adenocarcinoma cells into non-cancerous cells. The methods include contacting a sample comprising adenocarcinoma cells with a compound having the formula (“Formula I”): CH₃—(CH₂)ₙ—CH═CH—(CH₂)ₘ—COOH) wherein n is an integer from 0 to 15, m is an integer from about 1 to 16, and the sum of m and n is an integer from 6 to 16, or a pharmaceutically acceptable ester, salt, amide, solvate, or metabolite thereof. Also disclosed are methods for treating adenocarcinoma in a subject. The methods include administering to the subject an effective amount of a compound having Formula I or a pharmaceutically acceptable ester, salt, amide, solvate, or metabolite thereof.

The present invention claims the benefit of U.S. Provisional Patent Application Serial No. 60/257,809, filed Dec. 23, 2000, which is hereby incorporated by reference.

### FIELD OF THE INVENTION

The subject invention is directed generally to methods for decreasing proliferation of cancer cells, or of inducing apoptosis of cancer cells, or of inducing differentiation of cancer cells into non-cancerous cells and to methods for treating adenocarcinoma in a subject.

### BACKGROUND OF THE INVENTION

Pancreatic cancer is one of the most enigmatic and aggressive malignant diseases facing oncologists (Parker et al., “Cancer Statistics. 1996,” CA Cancer J. Clin., 46:5-27 (1996) (“Parker”)). It is now the fourth leading cause of cancer death in both men and women in the United States, and the incidence of this disease has significantly increased over the past 20 years (Parker; Trede et al., “Survival After Pancreaticoduodenectomy: 118 Consecutive Resections Without an Operative Mortality,” Ann. Surg., 211:447-458 (1990); Cameron et al., “One Hundred and Forty-five Consecutive Pancreaticoduodenectomies Without Mortality,” Ann. Surg., 217:430-438 (1993); Horward, “Pancreatic Adenocarcinoma,” Curr. Prob. in Cancer, 20:286-293 (1996) (“Horward”); Poston et al., Gut. Biology of Pancreatic Cancer, 32:800-812 (1991) (“Poston”); and Black et al., “Treatment of Pancreatic Cancer: Current Limitations, Future Possibilities,” Oncology, 10:301-307 (1996) (“Black”)). Pancreatic cancer is responsible for 27,000 deaths per year in the United States. Because of lack of early diagnosis and poor therapeutic responsiveness of pancreatic cancer, less than 2% of patients survive beyond five years, and the median expectation of life after diagnosis of pancreatic cancer is less than 6 months (Horward; Poston; and Black).

Colonic cancer is the second most common form of cancer in the United States (Doll et al., “Mortality in Relation to Smoking: 20 Years' Observations on Male British Doctors,” BMJ, 2:1525-1536 (1976); Hruban et al., “Molecular Diagnosis of Cancer and Micrometastases,” Adv. Anat. Pathol., 5:175-178 (1998) (“Hruban”); Figueredo et al., “Adjuvant Therapy for Stage II Colon Cancer After Complete Resection. Provincial Gastrointestinal Disease Site Group,” Cancer Prev. Control, 1:379-92 (1997) (“Figueredo”); Ness et al., “Outcome States of Colorectal Cancer: Identification and Description Using Patient Focus Groups,” Am. J. Gastroenterol., 93:1491-7 (1998) (“Ness”); Trehu et al., “Cost of Screening for Colorectal Cancer: Results of a Community Mass Screening Program and Review of Literature,” South Med. J., 85:248-253 (1992); and Wingo et al., “Cancer Statistics,” CA Cancer J. Clin., 45:8-30 (1995) (“Wingo”)). Colonic cancer occurs in more than 138,000 patients and is responsible for more than 55,000 deaths in the United States each year (Wingo). Up to 70% of patients with colonic cancer develop hepatic metastasises by the time of death, indicating that non-detectable detectable micro-metastases are present at the time of surgery (Hruban; Figueredo; and Ness). Furthermore, metastatic cancer is often not responsive to standard chemotherapeutic regimens, resulting in treatment failure (Figueredo and Ness). The overall response of advanced or non-resectable colorectal cancer patients to chemotherapeutic agents varies from 26 to 44 percent. For example, less than one third of colorectal cancer patients with liver metastases respond to treatment with agents such as 5-FU and leucovorin (Id.).

Breast cancer has the highest incidence of any cancer in women with the diagnosis being made in more than 275,000 per year in the USA (Richards et al., “Influence of Delay on Survival in Patients with Breast Cancer: A Systematic Review,” Lancet, 353:1119-26 (1999); Norton, “Adjuvant Breast Cancer Therapy: Current Status and Future Strategies—Growth Kinetics and the Improved Drug Therapy of Breast Cancer,” Semin. Oncol., 26:1-4 (1999); Morrow et al., “Current Controversies in Breast Cancer Management,” Curr. Probl. Surg., 36:163-216 (1999); and Ruppert et al., “Gene Therapy Strategies for Carcinoma of the Breast,” Breast Cancer Res. Treatment, 44:93-114 (1997)). Even though five year survival has increased to more than 80%, more than 77,000 women still die from this disease each year (Id.).

Thus, another dimension in chemotherapeutic agents for pancreatic, colonic, and breast cancer would be extremely beneficial, especially to control metastatic and unresectable disease.

### SUMMARY OF THE INVENTION

The present invention relates to a method of decreasing proliferation of adenocarcinoma cells, or of inducing apoptosis of adenocarcinoma cells, or of inducing differentiation of adenocarcinoma cells into non-cancerous cells. The method includes contacting a sample comprising adenocarcinoma cells with a compound having the formula:

CH₃—(CH₂)ₙ—CH═CH—(CH₂)ₘ—COOH

wherein n is an integer from 0 to 15, m is an integer from about 1 to 16, and the sum of m and n is an integer from 6 to 16, or a pharmaceutically acceptable ester, salt, amide, solvate, or metabolite thereof.

The present invention also relates to a method of treating adenocarcinoma in a subject. The method includes administering to the subject an effective amount of a compound having the formula:

CH₃—(CH₂)ₙ—CH═CH—(CH₂)ₘ—COOH

wherein n is an integer from 0 to 15, m is an integer from about 1 to 16, and the sum of m and n is an integer from 6 to 16, or a pharmaceutically acceptable ester, salt, amide, solvate, or metabolite thereof.

### BRIEF DESCRIPTION OF THE DRAWINGS

FIGS. 1A and 1B are bar graphs showing the effects of myristoleic acid obtained from Sigma Chemicals (St. Louis, Mo.) (FIG. 1A) and myristoleic acid obtained from Matreya, Inc. (State College, Pa.) (FIG. 1B) on the proliferation of AsPC-1 human pancreatic cancer cells.

FIGS. 2A and 2B are bar graphs showing the effects of impure cetyl myristoleic acid (FIG. 2A) and pure palmitoleic acid (FIG. 2B) on the proliferation of AsPC-1 human pancreatic cancer cells.

FIGS. 3A and 3B are bar graphs showing the effects of myristoleic acid on the proliferation of AsPC-1 (FIG. 3A) and PANC-1 (FIG. 3B) human pancreatic cancer cells.

FIG. 4 is a set of four fluorescence micrograph images showing the effect of myristoleic acid on annexin V binding.

FIGS. 5A and 5B are dot plots showing TUNEL assay results of AsPC-1 cells pancreatic cancer cells treated with 10 μg/ml of myristoleic acid for 24 hours (FIG. 5B) as compared to control (FIG. 5A).

FIG. 6 is an image of human pancreatic cancer xenografts in athymic mice which were treated with 250 mg/kg/day of myristoleic acid (labeled “myristoleic acid”) or control solution (labeled “control”).

FIGS. 7A and 7B are graphs showing the effects of myristoleic acid on tumor volume of subcutaneous xenografts of AsPC-1 (FIG. 7A) and HPAC (FIG. 7B) human pancreatic cancer cells in athymic mice as a function of time.

FIGS. 8A and 8B are bar graphs showing the effects of myristoleic acid on tumor weight of subcutaneous xenografts of AsPC-1 (FIG. 8A) and HPAC (FIG. 8B) human pancreatic cancer cells in athymic mice at the end of the experiment.

FIGS. 9A-9D are images produced with an in situ TUNEL assay conducted on sections of AsPC-1 pancreatic cancer cell xenografts harvested from athymic mice that were treated with control solution (vehicle only) (FIGS. 9A and 9B) or myristoleic acid (FIGS. 9C and 9D).

### DETAILED DESCRIPTION OF THE INVENTION

The present invention relates to a method of decreasing proliferation of adenocarcinoma cells, or of inducing apoptosis of adenocarcinoma cells, or of inducing differentiation of adenocarcinoma cells into non-cancerous cells. The method includes contacting a sample comprising adenocarcinoma cells with a compound having the formula (“Formula I”)

CH₃—(CH₂)ₙ—CH═CH—(CH₂)ₘ—COOH

wherein n is an integer from 0 to 15, m is an integer from about 1 to 16, and the sum of m and n is an integer from 6 to 16, or a pharmaceutically acceptable ester, salt, amide, solvate, or metabolite thereof.

Examples suitable compounds having Formula I include those in which m is 1 and n is 5, 6, 7, 8, 9, 10, 11, 12, 13, 14, or 15; in which m is 2 and n is 4, 5, 6, 7, 8, 9, 10, 11, 12, 13, or 14; in which m is 3 and n is 3, 4, 5, 6, 7, 8, 9, 10, 11, 12, or 13; in which in which m is 4 and n is 2, 3, 4, 5, 6, 7, 8, 9, 10, 11, or 12; in which m is 5 and n is 1, 2, 3, 4, 5, 6, 7, 8, 9, 10, or 11; m is 6 and n is 0, 1, 2, 3, 4, 5, 6, 7, 8, 9, or 10; m is 7 and n is 0, 1, 2, 3, 4, 5, 6, 7, 8, or 9; m is 8 and n is 0, 1, 2, 3, 4, 5, 6, 7, or 8; m is 9 and n is 0, 1, 2, 3, 4, 5, 6, or 7; m is 10 and n is 0, 1, 2, 3, 4, 5, or 6; m is 11 and n is 0, 1, 2, 3, 4, or 5; m is 12 and n is 0, 1, 2, 3, or 4; m is 13 and n is 0, 1, 2, or 3; m is 14 and n is 0, 1, or 2; m is 15 and n is 0 or 1; and m is 16 and n is 0. Preferably, when m is 7, n is 0, 1, 2, 3, 4, 5, 6, 8, or 9.

As indicated above, pharmaceutically acceptable base addition salts of the compounds of Formula I can also be used. Such salts include those derived from inorganic bases, such as ammonium and alkali and alkaline earth metal hydroxides, carbonates, bicarbonates, and the like, as well as salts derived from basic organic amines, such as aliphatic and aromatic amines, aliphatic diamines, hydroxy alkylamines, and the like. Such bases useful in preparing the salts useful in the practice of the present invention thus include ammonium hydroxide, potassium carbonate, sodium bicarbonate, calcium hydroxide, methyl amine, diethyl amine, ethylene diamine, cyclohexylamine, ethanolamine, and the like.

Pharmaceutically acceptable esters and amides of the compound of Formula I can also be employed in the method of the present invention. Examples of suitable esters include alkyl, aryl, and aralkyl esters, such as methyl esters, ethyl esters, propyl esters, dodecyl. esters, benzyl esters, and the like. Examples of suitable amides include, unsubstituted amides, monosubstituted amides, and disubstituted amides, such as methyl amide, dimethyl aminde, methyl ethyl amide, and the like.

In addition, the method of the present invention can be practiced using solvate forms of the compounds of Formula I or salts, esters, amides, and/or metabolies thereof, such as ethanol solvates, hydrates, and the like.

It is recognized that the compounds of Formula I can be in the cis or trans configuration. The method of the present invention can be practiced with pure cis isomer, pure trans isomer, a racemic mixture of cis and trans isomers, or any other mixture of cis and trans isomers.

Illustrative compounds which can be used in the practice of the method of the present invention include myristoleic acid or a pharmaceutically acceptable salt, ester (e.g., a cetyl ester), amide, metabolite, and/or solvate thereof.

Compounds of Formula I can be synthesized by established methods, such as those set forth in Beilstein 2(2) 423.

As explained above, the above-identified compounds can be used to decrease proliferation of adenocarcinoma cells, and/or induce apoptosis of adenocarcinoma cells, and/or induce differentiation of adenocarcinoma cells into non-cancerous cells. The meaning of the terms “proliferation”, “apoptosis”, and “differentiation” are readily understood in the art. Illustrative methods for assaying for proliferation, apoptosis, or differentiation are provided in the examples which follow and are also described in applicant's copending U.S. patent application Ser. No. 09/111,343, which is hereby incorporated by reference.

“Adenocarcinoma cells”, as used herein, are meant to include cancerous epithelial cells, such as prostate cancer cells, lung cancer cells, stomach cancer cells, breast cancer cells, pancreatic cancer cells, and colon cancer cells. The methods of the present invention can be practiced in vitro or in vivo.

More particularly, the method of the present invention can be used in vivo to treat adenocarcinomas, such as prostate cancer, lung cancer, stomach cancer, pancreatic cancer, breast cancer, and colon cancer. In the case where the method of the present invention is carried out in vivo, for example, where the adenocarcinoma cells are present in a human subject, contacting can be carried out by administering a therapeutically effective amount of the compound to the human subject, for example, by directly injecting the compound into a tumor. Details with regard to administering compounds in accordance with the method of the present invention are described below.

The present invention, in another aspect thereof, relates to a method of treating adenocarcinomas, such as prostate cancer, lung cancer, stomach cancer, breast, pancreatic cancer, colon cancer, esophageal cancer, uterine cancer, ovarian cancer, or other cancers involving epithelial cells. The method includes administering, to the subject, a compound of Formula I or a pharmaceutically acceptable ester, salt, amide, solvate, or metabolite thereof.

Suitable subjects include, for example mammals, such as rats, mice, cats, dogs, monkeys, and humans. Suitable human subjects include, for example, those which have previously been determined to be at risk of having prostate cancer, lung cancer, stomach cancer, pancreatic cancer, colon cancer, and/or breast cancer and those who have been diagnosed as having prostate cancer, lung cancer, stomach cancer, pancreatic cancer, colon cancer, and/or breast cancer. Preferably, the subject suffers from only one of these types of cancers, for example, from only pancreatic cancer.

In subjects who are determined to be at risk of having adenocarcinoma, the above-identified compounds of Formula I or salts, esters, amides, solvates, and metabolites thereof are administered to the subject, preferably under conditions effective to decrease proliferation and/or induce apoptosis and/or induce differentiation of the adenocarcinoma cells in the event that they develop. Such preventive (which is not used in the absolute 100% sense) therapy can be useful in high risk individuals as long as the adverse side effects of the administration of these compounds are outweighed by the potential benefit of prevention.

Any of the compounds described above can be used in the treatment method of the present invention. For example, compounds may be administered alone or in combination with compatible carriers as a composition. Compatible carriers include suitable pharmaceutical carriers or diluents. The diluent or carrier ingredients should be selected so that they do not diminish the therapeutic effects of the compounds used in the present invention.

The compositions herein may be made up in any suitable form appropriate for the desired use. Examples of suitable dosage forms include oral, parenteral, or topical dosage forms.

Suitable dosage forms for oral use include tablets, dispersible powders, granules, capsules, suspensions, syrups, and elixirs. Inert diluents and carriers for tablets include, for example, calcium carbonate, sodium carbonate, lactose, and talc. Tablets may also contain granulating and disintegrating agents, such as starch and alginic acid; binding agents, such as starch, gelatin, and acacia; and lubricating agents, such as magnesium stearate, stearic acid, and talc. Tablets may be uncoated or may be coated by known techniques to delay disintegration and absorption. Inert diluents and carriers which may be used in capsules include, for example, calcium carbonate, calcium phosphate, and kaolin. Suspensions, syrups, and elixirs may contain conventional excipients, for example, methyl cellulose, tragacanth, sodium alginate; wetting agents, such as lecithin and polyoxyethylene stearate; and preservatives, such as ethyl-p-hydroxybenzoate.

Dosage forms suitable for parenteral administration include solutions, suspensions, dispersions, emulsions, and the like. They may also be manufactured in the form of sterile solid compositions which can be dissolved or suspended in sterile injectable medium immediately before use. They may contain suspending or dispersing agents known in the art.

Examples of parenteral administration are intraventricular, intracerebral, intramuscular, intravenous, intraperitoneal, rectal, and subcutaneous administration.

In addition to the above, generally non-active components of the above-described formulations, these formulations can include other active materials, particularly, actives which have been identified as useful in the treatment of prostate, lung, stomach, breast, colon, pancreatic cancers and/or other adenocarcinomas. These actives can be broad-based anti-cancer agents, such that they also are useful in treating other types of cancers (i.e., in addition to adenocarcinomas) or they may be more specific, for example, in the case where the other active is useful for treating adenocarcinomas or particular types of adenocarcinomas. The other actives can also have non-anti-cancer pharmacological properties in addition to their anti-adenocarcinoma properties. For example, the other actives can have anti-inflammatory properties, or, alternatively, they can have no such anti-inflammatory properties.

It is understood that some of the compounds described above (i.e., some of the compounds that are useful in the methods of the present invention) are naturally occurring. The compositions used in the treatment method of the present invention can be substantially free of one or more of the components with which the compound is typically found when it is naturally occurring. Alternatively or additionally, the compositions used in the treatment method of the present invention can be substantially free of all but one of the components with which the compound is typically found when it is naturally occurring. Still alternatively or additionally, the compositions used in the treatment method of the present invention can be substantially free of all of the components with which the compound is typically found when it is naturally occurring. For the purposes of the present application, a composition is considered to be substantially free of component X when the amount of component X is less than 10% by weight (such as less than 5% by weight, less than 2% by weight, and/or less than 1% by weight) relative to the weight of the composition.

It will be appreciated that the actual preferred amount of compound to be administered according to the present invention will vary according to the particular compound, the particular composition formulated, and the mode of administration. Many factors that may modify the action of the compound (e.g., body weight, sex, diet, time of administration, route of administration, rate of excretion, condition of the subject, drug combinations, and reaction sensitivities and severities) can be taken into account by those skilled in the art. Administration can be carried out continuously or periodically within the maximum tolerated dose. Optimal administration rates for a given set of conditions can be ascertained by those skilled in the art using conventional dosage administration tests.

The present invention is further illustrated with the following examples.

### EXAMPLES

### Example 1

### Effect of Myristoleic Acid on Proliferation of Human Pancreatic Cancer Cells

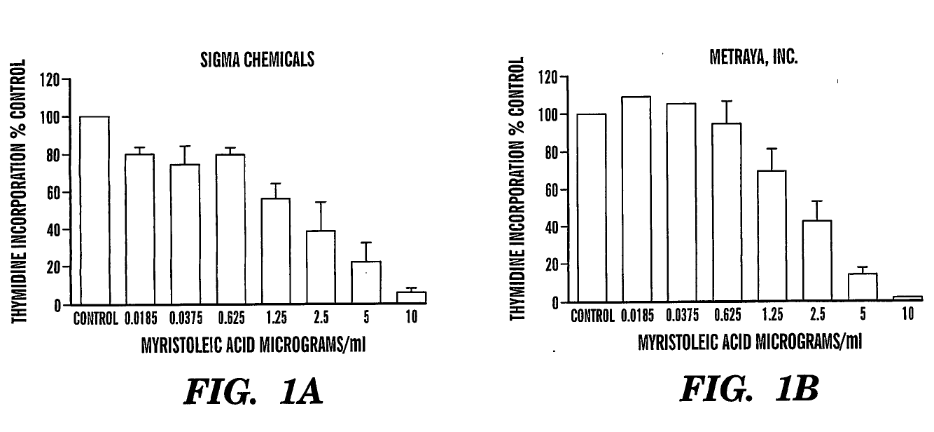

Myristoleic acid, obtained from two separate commercial sources, caused a concentration-dependent inhibition of proliferation of AsPC-1 human pancreatic cancer cells as measured by thymidine incorporation at 24 hours. This can be seen in FIG. 1A (results using myristoleic acid obtained from Sigma Chemicals (St. Louis, Mo.)) and FIG. 1B (results using myristoleic acid obtained from Matreya, Inc. (State College, Pa.)).

### Example 2

### Effect of Cetyl Myristoleic Acids and Palmitoleic Acid on Proliferation of Human Pancreatic Cancer Cells

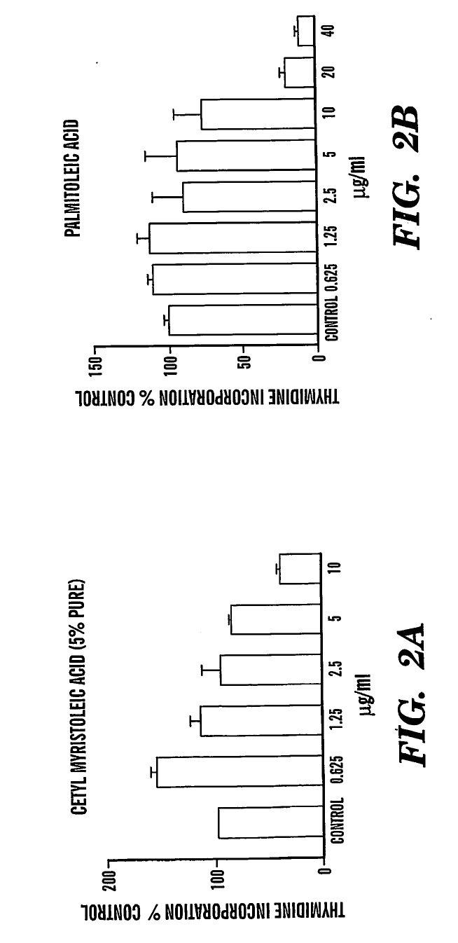

Impure cetyl myristoleic acid (approximately 5% pure) and pure palmitoleic acid also caused a concentration-dependent inhibition of proliferation of AsPC-1 human pancreatic cancer cells as measured by thymidine incorporation. The results are presented in FIG. 2A (cetyl myristoleic acid) and FIG. 2B (palmitoleic acid). As FIGS. 2A and 2B show, the effects of impure cetyl myristoleic acid and palmitoleic acid on AsPC-1 human pancreatic cancer cells proliferation are similar to, though less potent than, the effects of myristoleic acid (shown in FIGS. 1A and 1B). Irrespective of whether myristoleic acid, cetyl myristoleic acid, or palmitoleic acid was used, the inhibition of proliferation was accompanied by morphological changes associated with apoptosis, including membrane blebbing, cellular rounding, and detachment from the culture plates (data not shown).

### Example 3

### Effect of Myristoleic Acid on Proliferation of AsPC-1 and PANC-1 Human Pancreatic Cancer Cells

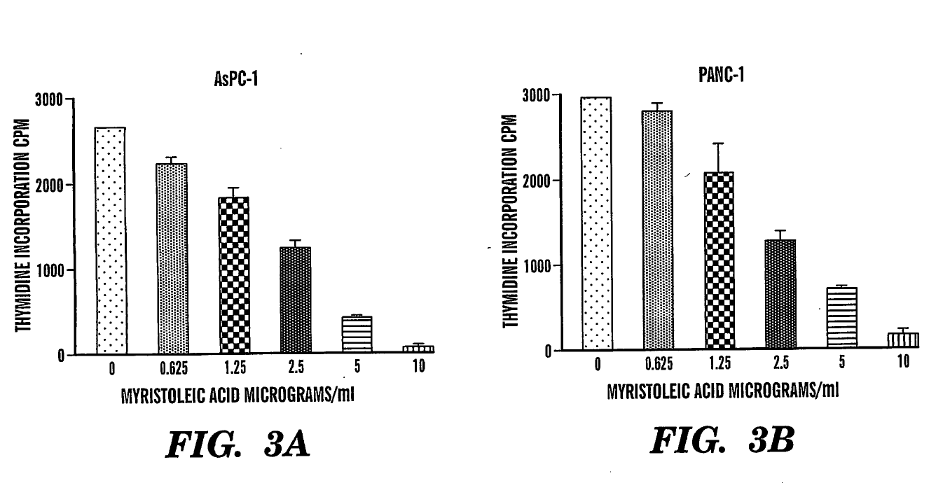

The thymidine incorporation experiments described in Example 1 were repeated in triplicate on each of two malignant human pancreatic cancer cell lines, AsPC-1 and PANC-1. The results are set forth in FIG. 3A (AsPC-1) and FIG. 3B (PANC-1). In each case, concentration-dependent effects of myristoleic acid on pancreatic cancer cell proliferation were observed. From these experiments it was determined that myristoleic acid has potent anti-proliferative activity in AsPC-1 and PANC-1 pancreatic cancer cells with effects in the low micromolar range.

### Example 4

### Effect of Myristoleic Acid on Annexin V Binding in AsPC-1 Human Pancreatic Cancer Cells

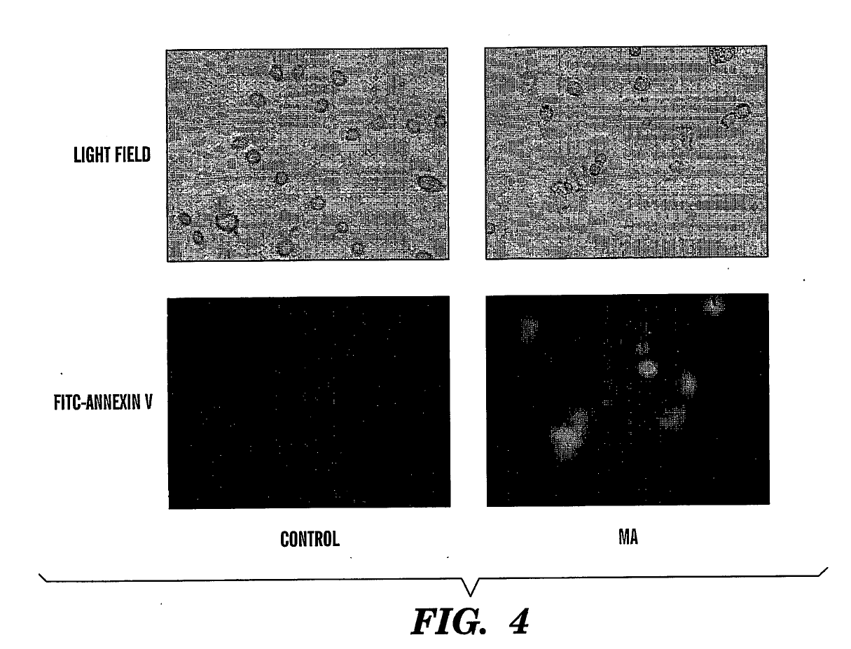

The effect of myristoleic acid (10 μg/ml) (“MA”) on annexin V binding in AsPC-1 cells at 3 or 5 hours after beginning treatment is shown in FIG. 4. Marked membrane fluorescence indicates early apoptosis in these cells. This specific test utilizes the translocation of phophatidyl serine from the inner to outer plasma membrane, which is a feature of early apoptosis. The results set forth in FIG. 4 are representative of three separate experiments.

### Example 5

### Effect of Myristoleic Acid on DNA Fragmentation by TUNEL Assay

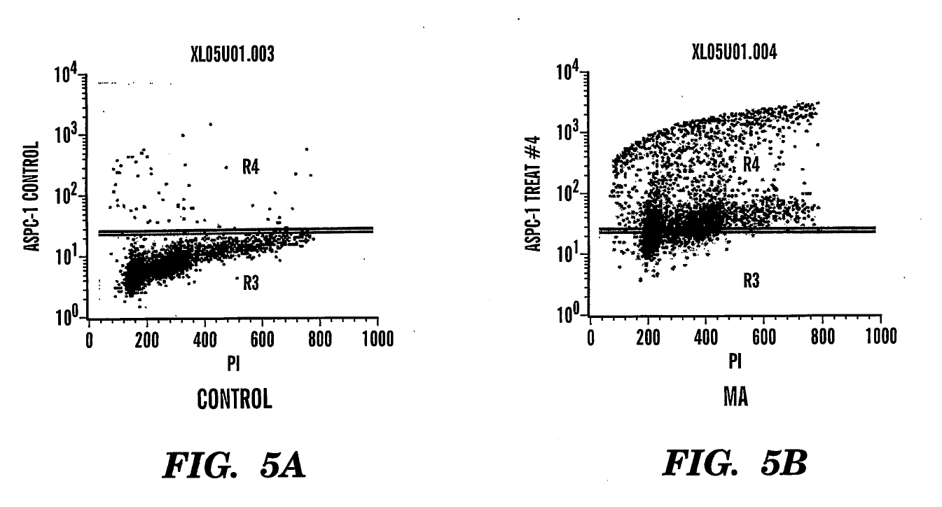

The effect of myristoleic acid (10 μg/ml) (“MA”) on terminal deoxynucleotidyl transferase-mediated dUTP nick end labeling (“TUNEL”) assay at 24 hours in AsPC-1 cells was investigated. The results are presented in FIGS. 5A (control) and 5B (10 μg/ml myristoleic acid). As FIGS. 5A and 5B demonstrate, 10 μg/ml myristoleic acid caused a marked increase in the number of apoptotic cells, shown in the upper right panel compared with control in the upper left. The results set forth in FIGS. 5A and 5B are representative of three separate experiments.

### Example 6

### Effect of Myristoleic Acid on Growth of Human Pancreatic Cancer Xenografts in Athymic Mice

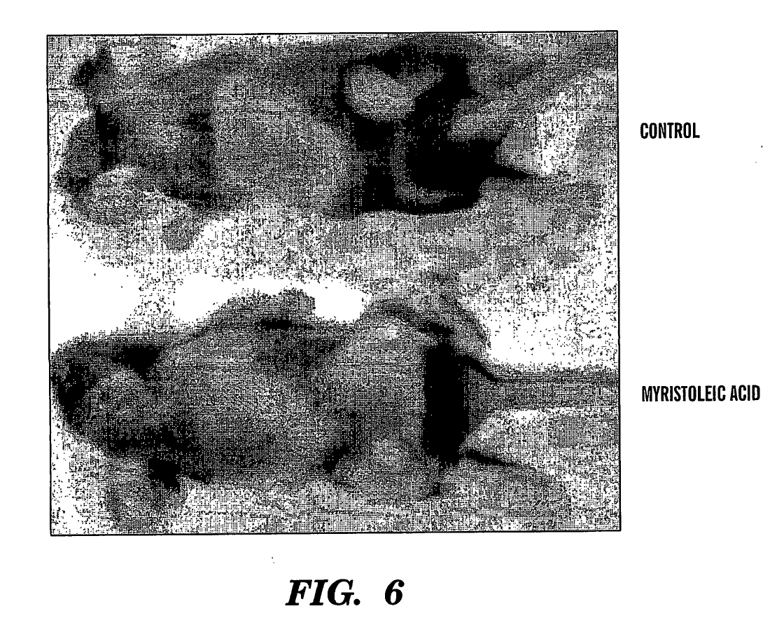

The effects of daily intraperitoneal injection of myristoleic acid (250 mg/kg/day) on the growth of AsPC-1 and HPAC xenografts in athymic mice were investigated. The results are presented in FIG. 6. Animals were injected daily with myristoleic acid (250 mg/kg/day) (labeled “myristoleic acid” in FIG. 6) or with a control solution (labeled “control” in FIG. 6) once visible tumors were established (about five days after implantation). As the sizes of the tumors in FIG. 6 demonstrate, myristoleic acid caused a marked reduction in tumor size throughout the experimental period. The experiment was repeated twice with AsPC-1 cells and also with HPAC cells. The results set forth in FIG. 6 are representative of these experiments.

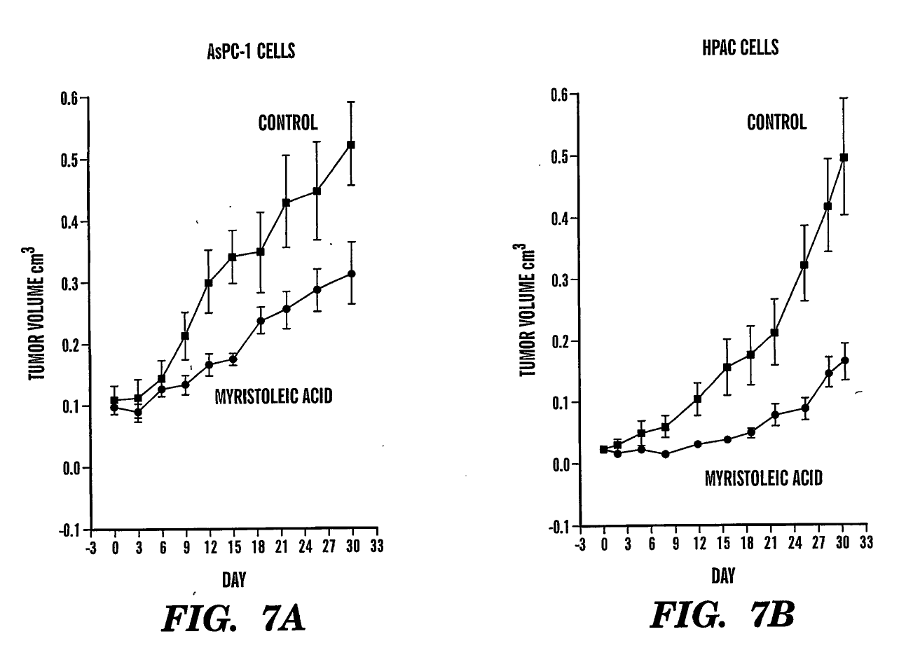

Tumor volumes of the above-described athymic mice were measured as a function of time, and the results are presented in FIG. 7A (AsPC-1 cancer cells) and FIG. 7B (HPAC cancer cells). FIGS. 7A and 7B demonstrate that myristoleic acid caused a marked reduction in both AspC-1 and HPAC tumor volume throughout the experimental period.

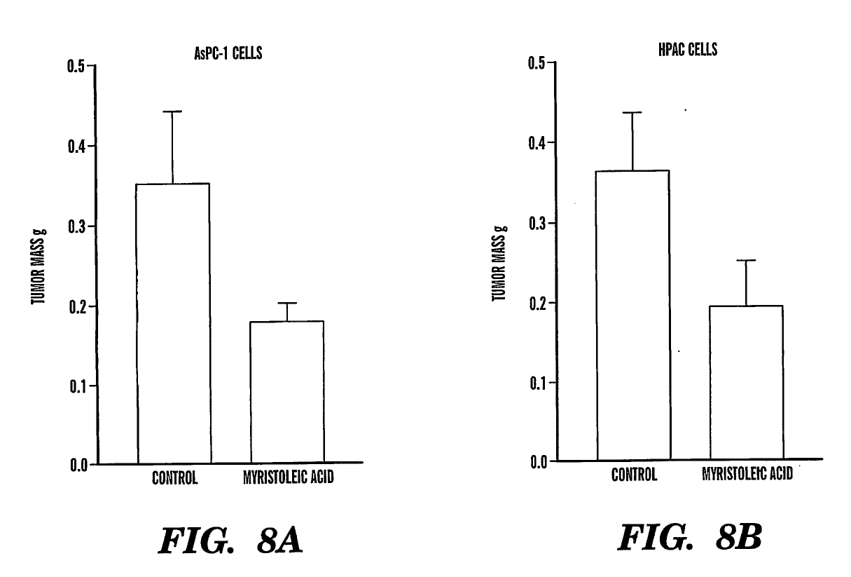

At the end of the experiment, the AsPC-1 and HPAC xenografts were harvested from the athymic mice and the weights of the tumors were measured. The results, presented in FIG. 8A (AspC-1 cancer cells) and FIG. 8B (HPAC cancer cells), show that myristoleic acid caused a marked reduction in AspC-1 and in HPAC tumor weight in all three experiments.

### Example 7

### In Situ TUNEL Assay of Human Pancreatic Cancer Xenografts in Athymic Mice

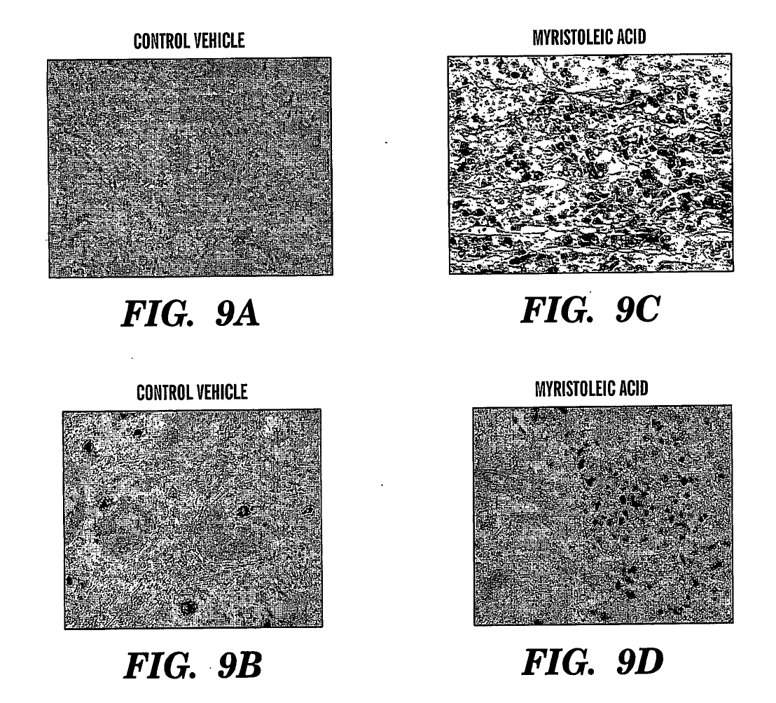

To determine whether the effects observed in the athymic mouse experiments described in Example 7 might be due to apoptosis, in situ TUNEL assays were carried out on the AsPC-1 xenografts that were harvested from the athymic mice at the end of the experiment. Images of xenograft sections from mice treated with the control solution (vehicle only) (FIGS. 9A and 9B) show minimal staining. In contrast, images of xenograft sections from mice treated with the myristoleic acid (FIGS. 9C and 9D) show many apoptotic cells (visualized by the dark brown staining). Thus, FIGS. 9A-9D demonstrate that cells in the myristoleic acid-treated tumor are undergoing apoptosis (programmed cell death).

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed is:

1. A method of decreasing proliferation of adenocarcinoma cells, or of inducing apoptosis of adenocarcinoma cells, or of inducing differentiation of adenocarcinoma cells into non-cancerous cells, said method comprising:
contacting a sample comprising adenocarcinoma cells with a compound having the formula:
CH₃—(CH₂)ₙ—CH═CH—(CH₂)ₘ—COOH
wherein n is an integer from 0 to 15, m is an integer from about 1 to 16, and the sum of m and n is an integer from 6 to 16, or a pharmaceutically acceptable ester, salt, amide, solvate, or metabolite thereof.

2. A method according to claim 1, wherein m is 7.

3. A method according to claim 1, wherein n is 3.

4. A method according to claim 1, wherein m is an integer from 6 to 8 and n is an integer from 2 to 4.

5. A method according to claim 1, wherein m is 7 and n is 3.

6. A method according to claim 1, wherein the compound is in the cis configuration.

7. A method according to claim 1, wherein m is 7 and n is 3, and wherein the compound is in the cis configuration.

8. A method according to claim 1, wherein the sample comprises prostate cancer cells, lung cancer cells, stomach cancer cells, breast cancer cells, pancreatic cancer cells, colon cancer cells, or combinations thereof.

9. A method according to claim 1, wherein the adenocarcinoma cells are present in a human subject and wherein said contacting comprises administering a therapeutically effective amount of the compound to the human subject.

10. A method of treating adenocarcinoma in a subject, said method comprising:
administering to the subject an effective amount of a compound having the formula:
CH₃—(CH₂)ₙ—CH═CH—(CH₂)ₘ—COOH
wherein n is an integer from 0 to 15, m is an integer from about 1 to 16, and the sum of m and n is an integer from 6 to 16, or a pharmaceutically acceptable ester, salt, amide, solvate, or metabolite thereof.

11. A method according to claim 10, wherein the subject is a human subject.

12. A method according to claim 10, wherein the amount is effective to decrease proliferation of cancer cells in the subject.

13. A method according to claim 10, wherein the amount is effective to induce apoptosis of cancer cells in the subject.

14. A method according to claim 10, wherein the amount is effective to induce differentiation of cancer cells in the subject into non-cancerous cells.

15. A method according to claim 10, wherein m is 7.

16. A method according to claim 10, wherein n is 3.

17. A method according to claim 10, wherein m is an integer from 6 to 8 and n is an integer from 2 to 4.

18. A method according to claim 10, wherein m is 7 and n is 3.

19. A method according to claim 10, wherein the compound is in the cis configuration.

20. A method according to claim 10, wherein the adenocarcinoma is selected from the group consisting of prostate cancer, lung cancer, stomach cancer, breast cancer, colon cancer, pancreatic cancer, and combinations thereof.
